(19) United States
(12) Patent Application Publication
(10) Pub. No.: US 2022/0249715 A1
(43) Pub. Date: Aug. 11, 2022

(54) UTILIZATION OF PHOTOCATALYTIC, PHOTOCHEMICALLYTIC AND DISSOCIATION REACTIONS IN COMBINATIONS WITH RADIATION AND OXIDIZING AGENTS

(71) Applicant: PHOTOCHEMICALLYTIC, LLC, Fort Worth, TX (US)

(72) Inventors: Marc W. GUNDERSON, Fort Worth, TX (US); Paul DABNEY, Georgetown, TX (US)

(73) Assignee: PHOTOCHEMICALLYTIC, LLC, Fort Worth, TX (US)

(21) Appl. No.: 17/165,127

(22) Filed: Feb. 2, 2021

(51) Int. Cl. A61L 2/08; A61L 2/10; A61L 2/26; C11D 3/395; A01N 59/00

(52) U.S. Cl. CPC A61L 2/088; A61L 2/10; A61L 2/084; A61L 2/085; A01N 59/00; C11D 3/3953; C11D 3/3955; C11D 3/3951; A61L 2/26

(57) ABSTRACT

Methods, systems, and apparatuses for producing one or more of trioxygen, reactive nitrogen species, hydrogen, oxygen, and electronically modified oxygen derivatives from oxidizing agents that are exposed to certain frequencies of radiation, exposed for certain amounts of time, and exposed to certain intensities of radiation. The oxidizing agent or oxidizing agents can be exposed to multiple frequencies of radiation and multiple exposures of radiation. A combination of one or more oxidizing agents and radiation of certain wavelengths forms a synergistic reaction. The synergistic reaction generates, among other agents, RNS, EMODs, which can further produce variation in the standard chemical reaction associated with the decomposition of the oxidizing agent. This reaction variation may produce RNS, trioxygen, hydrogen and/or its isotopes, and/or oxygen and/or its isotopes and/or electronically modifies oxygen derivatives. This synergistic reaction has a relationship to EMOD creation, Oxygen and its isotope generation and hydrogen and its isotope generation.

### BACKGROUND

Oxidizing agents such as hydrogen peroxide have received increasing attention as an energy carrier. To achieve sustainable energy, photocatalytic splitting of oxidizing agents such as hydrogen peroxide is a desirable reaction for on-site hydrogen generation. Numerous applications across many industries have been found. From energy storage and production, medical, food, environmental and others, this previously unknown method of combining, photocatalytic, photochemicallytic and dissociation reactions with radiation and oxidizing agents opens a new frontier. This reaction has not previously been reported because conventional photocatalysis decomposes oxidizing agents by disproportionation and by promoting oxidizing agent reduction instead of hydrogen liberation. Here we report the successful example of oxidizing agent splitting. Trioxygen associates with the reactants and suppresses the reactant reduction, thus promoting hydrogen liberation. The organic photocatalytic system may provide a basis of photocatalytic and photochemicallytic oxidizing agent splitting. An oxidizing agent is a chemical species that undergoes a chemical reaction in which it gains one or more electrons. Also, an oxidizing agent can be regarded as a chemical species that transfers electronegative atoms, usually oxygen, to a substrate. The oxidizing agent can be called an oxygenation reagent or oxygen-atom transfer (OAT) agent. Oxidation reactions may involve oxygen atom transfer reactions and hydrogen atom abstraction which is a reaction where removal of an atom or group from a molecule by a radical occurs. The UV radiation commonly used in antimicrobial processes is known as UV-C. Ultra-Violet (UV) light is invisible to the human eye and is divided into UV-A, UV-B and UV-C. UV-C is found within 100-280 nm range. The germicidal action of UV-C is maximized at approximately 265 nm with reductions on either side. UV-C sources typically have their main emission at 254 nm.

As a result, germicidal lamps can be effective in breaking down the DNA of microorganisms. This means that they cannot replicate and cause disease. UV radiation also can be used to produce or eliminate trioxygen which can be a hazardous Reactive Oxygen Species. Reactive nitrogen species (RNS) is a subset of free oxygen radicals called reactive oxygen species (ROS). Trioxygen can also be used as a catalyst to convert H2O to products that exhibit health benefits, antimicrobial properties and have wide commercial uses.

In chemistry, photocatalysis is the acceleration of a photoreaction in the presence of a catalyst. Photocatalysts are materials that change the rate of a chemical reaction on exposure to light. In catalyzed photolysis, light is absorbed by a substrate. Photocatalytic activity (PCA) depends on the ability of the catalyst to create electron-hole pairs, which utilize electronically modified oxygen derivatives which are then able to undergo secondary reactions. Typically, two types of photocatalysis reactions are recognized, homogeneous photocatalysis and heterogeneous photocatalysis. Homogeneous photocatalysis: when both the photocatalyst and reactant are in the same phase, i.e. gas, solid, or liquid, such photocatalytic reactions are termed as homogeneous photocatalysis. Heterogeneous photocatalysis: when both the photocatalyst and reactant are in different phases, such photocatalytic reactions are classified as heterogeneous photocatalysis. When a photocatalyst is exposed to radiation of the desired wavelength (sufficient energy), the energy of photons is absorbed by an electron (e−) of valence band and it is excited to conduction band. In this process a hole (h+) is created in valence band. This process leads to the formation of photo-excitation state, and e− and h+ pair is generated. The hydroxyl radical is generated in both types of reaction. The difference in the two types of photocatalytic reactions are the placements of the reactants and the photocatalysts

### SUMMARY

In one embodiment, a method for generating photo oxidation products, photocatalytic products and/or photochemicallytic products which include one or more of reactive nitrogen species, hydrogen and its isotopes, oxygen and its isotopes, and electronically modifies oxygen derivatives, reactive oxygen species, trioxygen, and free radicals, may be provided and may include applying at least one oxidizing agent to a target; and before, and/or during, and/or after the at least one oxidizing agent is applied to the target, applying radiation to the oxidizing agent, which forms a synergistic reaction and produces the photo oxidation products, where the photo oxidation products comprise at least trioxygen and hydroxyl radical, and wavelengths that photodissociate, eliminate, or reduce trioxygen are excluded from the radiation.

In another embodiment, a system for performing the steps of the above method may be provided. The system can include a reaction area, in which the at least one oxidizing agent functions together with the radiation of certain wavelengths to lead to a synergistic reaction, so that the products of the reaction can be collected and separated any time during the reaction if desired, at least one oxidizing agent introducing component for applying the at least one oxidizing agent to the target, and at least one radiation emitting component for creating the radiation wherein wavelengths that can photodissociate, eliminate, or reduce trioxygen are excluded from the radiation.

### BRIEF DESCRIPTION OF THE DRAWINGS

Advantages of exemplary embodiments of the system, retainer and method of providing therapeutic treatment will be apparent from the following detailed description of the exemplary embodiments. The following detailed description should be considered in conjunction with the accompanying figures in which:

FIG. 1 is an exemplary diagram showing a reaction can occur from a reactant molecule via an intermediate such as hydroperoxyl to form an trioxygen molecule.

FIG. 2 is an exemplary diagram showing a “stored” oxidizing effect that can be tapped to provide reactive oxygen species as needed, and the “stored” oxidizing effect feeds the looped chain reaction so that reactive oxygen species are generated until one of the reactants is depleted.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

In the system displayed herein, a unique method of utilizing both homogeneous and heterogeneous reactions is described. By utilizing both types of PCA, a self-sustaining reaction is produced resulting in electronically modified oxygen derivatives that are continuously produced as long as reactants are present. Trioxygen is one of the potential photocatalysts. This results in an increased efficacy and a shelf life of increased and sustainable reactivity previously not producible with oxidizing agents.

The embodiments relate to producing one or more of reactive nitrogen species, trioxygen, hydrogen and/or its isotopes, and/or oxygen and/or its isotopes and/or electronically modifies oxygen derivatives, reactive oxygen species, free radicals, oxidizing molecules, oxygen-atom transfer (OAT) agents, oxidizing agents and/or various related species from oxidizing agents that are exposed to certain frequencies of radiation, exposed for certain amounts of time and exposed to certain intensities of radiation. The oxidizing agents can be exposed to multiple frequencies of radiation and multiple exposures of radiation. The radiation can be supplied to the oxidizing agents continuously or in bursts or pulses. During research into the effects of radiation on oxidizing agents, a discovery was made that offers a revolutionary and multi-disciplinary advancement to science. The methods displayed provide a new paradigm to perform photocatalytic oxidation of substrates using radiation as energy input, trioxygen as the catalyst and oxidizing agents as the oxygen source and dissociation reactions to minimize hindrances to the reactions. Understanding the chemistry of this new paradigm is essential for utilizing the reactivity. Photocatalytic activity (PCA) is commonly applied to a target in two distinct ways.

Further, the embodiments utilize both methods of applying photocatalytic activity to generate a unique reaction that continues even after the radiation that initiates the PCA is discontinued. The present research, which is reflected in the embodiments, explored PCA utilizing radiation and a significant discovery was made. It has now been found that the destruction of trioxygen (O3) by certain wavelengths of radiation prevents or retards reactions involved in the photocatalytic effects. The catalyst, trioxygen, was being eliminated by certain wavelengths of radiation that encourage dissociation. By altering the production or availability of trioxygen, the reaction may include steps that allows and encourages or alternatively prevents or retards the generation of products such as oxygen and hydrogen, reactive nitrogen species, electronically modified oxygen derivatives and others.

In one exemplary embodiment, it may be understood that after trioxygen is produced it will decay rapidly, because trioxygen is an unstable compound with a relatively short half-life. The half-life of trioxygen in liquid is a lot shorter than in air. Trioxygen decays in liquids partly in reactions with hydroxyl radicals. The assessment of a trioxygen decay process always involves the reactions of two species: trioxygen and hydroxyl radicals. When these hydroxyl radicals are the dominant particles in the solution, it is called an advanced oxidation process (AOP). The decay of trioxygen in contact with hydroxyl radicals in liquids is characterized by a fast initial decrease of trioxygen, followed by a second phase in which trioxygen decreases by first order kinetics. Dependent on the quality of the liquids, the half-life of trioxygen is in the range of seconds to hours in common testing. Factors influencing the decomposition of trioxygen in liquids are temperature, pH, ions, cations, environment and concentrations of dissolved matter and UV light.

As mentioned above, trioxygen decomposes partly in hydroxyl radicals. When the pH value increases, the formation of hydroxyl radicals increases. In a solution with a high pH value, there are more hydroxide ions present, see formulas 0-1 and 0-2 below. These hydroxide ions act as an initiator for the decay of trioxygen:

O₃+OH⁻→HO₂⁻+O₂ Equation 0-1

O₃+HO₂—→^{•}OH+O₂^{•-}+O₂ Equation 0-2

The radicals that are produced during reaction 0-2 can introduce other reactions with trioxygen, causing more hydroxyl radicals to be formed. Dependent on the nature of dissolved matter in a liquid, these can accelerate or slow down the decay of trioxygen. Substances that accelerate this reaction are called promoters. Inhibitors are substances that slow down the reaction. When a liquid is infused with trioxygen, one often uses the term ‘scavenging capacity’ in reference to the decay rate of the trioxygen. Scavengers and inhibitors are entities that react with hydroxyl radicals and slow down the reaction between trioxygen and hydroxyl radicals. Some common methods of inhibiting the decay of trioxygen involve lowering the pH of the target liquid and using deionized solutions as dilutants when possible.

In further exemplary embodiments, oxidative reactions due to photocatalytic homogenous effect may be described and utilized as follows:

The mechanism of hydroxyl radical production can follow paths such as:

O₃+hv→O₂+O Equation 1

O+H₂O→•OH+•OH Equation 2

O+H₂O→H₂O₂ Equation 3

H₂O₂+hv→•OH+•OH Equation 4

Similarly, the Fenton system produces hydroxyl radicals by the following mechanism:

Fe²⁺+H₂O₂→HO•+Fe³⁺+OH⁻ Equation 5

Fe³⁺+H₂O₂→Fe²⁺+HO•2+H⁺ Equation 6

Fe²⁺+HO•→Fe³⁺+OH⁻ Equation 7

In photo-Fenton type processes, additional sources of OH radicals should be considered: through photolysis of H₂O₂, and through reduction of Fe³⁺ ions under radiation:

H₂O₂+radiation→HO•+HO• Equation 8

Fe³⁺+H₂O+radiation→Fe²⁺+HO•+H⁺ Equation 9

Oxidative reactions due to photocatalytic heterogenous effect:

h⁺+H₂O→H++•OH Equation 10

2h⁺+2H₂O→2H⁺+H₂O₂ Equation 11

H₂O₂→2•OH Equation 12

The reaction of H2O2=H2O+O is typically referenced in most literature as the predominant disassociation reaction associated with hydrogen peroxide and results in the production of oxygen and water. There are a number of reaction pathways such as dissociation to hydronium ion and hydroperoxide, and disproportionation to dioxygen and water. Note that TRIOXYGEN is not produced in the above reactions. Trioxygen is photodissociated by certain wavelengths of radiation. While trioxygen may be created, it may also be dissociated depending on the desired outcome of the reaction. The table below is a partial list of the products of trioxygen dissociation and a partial list of the wavelengths associated with those products.

TABLE 1

| | | |
|---|---|---|
| O(³P) + O₂(³Σ) | 1118.4 | nm |
| O(³P) + O₂(¹Δ) | 599.2 | nm |
| O(³P) + O₂(¹Σ) | 452.6 | nm |
| O(¹D) + O₂(³Σ) | 402.8 | nm |
| O(¹D) + O₂(¹Δ) | 307.0 | nm |
| O(¹D) + O₂(¹Σ) | 263.3 | nm |
| O(³P) + O(³P) + O(³P) | 197.1 | nm |

In one path, the embodiments describe one or more reactions whereby the trioxygen is not photodissociated by radiation. Trioxygen then becomes a photocatalyst for newly discovered reactions. The resulting reaction is one that has not previously been described. Trioxygen is produced and retained when the above-mentioned wavelengths of photodissociation are excluded. This exclusion coupled with photocatalytic reactions generating one or more of reactive nitrogen species, trioxygen, hydrogen and/or its isotopes, and/or oxygen and/or its isotopes and/or electronically modifies oxygen derivatives, reactive oxygen species, free radicals, oxidizing molecules, oxidizing agents and/or various related species from oxidizing agents that are exposed to certain frequencies of radiation. The reaction with OH— is the initial decomposition step of trioxygen decay, the stability of a trioxygen solution is thus highly dependent on pH and decreases as alkalinity rises. At pH above 8 the initiation rate has, in the presence of radical scavengers, been shown to be proportional to the concentrations of trioxygen and OH—. However, in acidic solutions the reaction with OH— is not the initiation step. Predicted reaction rates below pH 4 including a mechanism based only on reaction with OH— are much lower than those determined experimentally. The trioxygen equilibrium reaction below becomes significant and the initiation reaction is catalyzed.

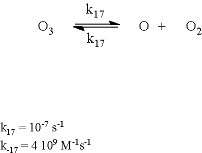

The atomic O continues to react with H2O, or forms an excited trioxygen radical, from recombination, that subsequently reacts with H2O, as shown in the two equations below, respectively.

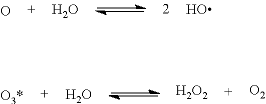

The species formed can then react further, forming other radicals such as O2-/HO2. The propagating products, HO• and HO2, diffuse and react with trioxygen in the continuing the chain reaction. Only low concentrations of the terminating species are present in the solution which is why the significant part of the termination reactions below also takes place.

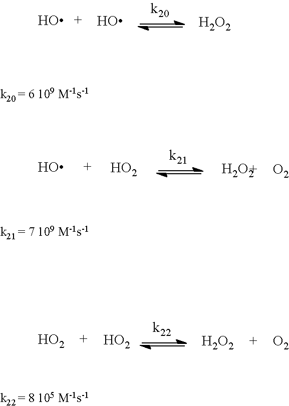

An example of an oxidizing agent involved in this reaction; H2O2+radiation between 100 nm and 1200 nm (where the wavelengths causing photodissociation of trioxygen have been excluded), when H2O2 and this selective radiation are combined, this reaction yields H2+2HO2 which in turn yields H2O+trioxygen. This looped chain reaction will continue as long as the correct radiation is present and H2O2 (oxidizing agent) is present. The 2 paths of this reaction can yield various products but particularly H2 and O2 or yield 2HO2. The trioxygen that is created on this path enters and exists in a looped chain reaction with H2O and the looped chain reaction will continue to function and is dependent on the supply of trioxygen or hydroperoxyls generated from reactions of trioxygen or hydroxyl radicals or generated from reactions of trioxygen with other reactants. A looped chain reaction includes numerous reactions and potential reactions that may vary depending on variables such as temperature, pH, catalysts, and others. The more basic and recognizable reaction is the looped chain reaction where it is trioxygen that reacts with water producing at various stages O2, hydroxyls, H2, HO3, HO4, and hydroperoxyls. Exposure of oxidizing agents such as hydrogen peroxide with the entire UV spectrum of radiation produces hydroxyl radicals but limited or no trioxygen due to the wavelengths that are present that also destroy trioxygen, which was previously undiscovered and, without this step, the products of this reaction could not be produced in a sustained looped chain reaction. Furthermore, if this step is performed, but performed in the wrong sequence, the reaction will not have the desired results and the sustained looped chain reaction will not occur. Hydroxyl radicals are very reactive free radicals, but they only exist for extremely brief periods of time measured in nana seconds. This nano second long existence leads to a short-term effect whereby the hydroxyl radicals exert an influence that cannot be stored or held in reserve. While this immediate effect has many uses, the production of trioxygen by the irradiation of oxidizing agents with radiation of certain wavelengths that exclude those wavelengths associated with the dissociation of trioxygen produces reactants such as hydroperoxyls that react to form trioxygen. With trioxygen in a looped chain reaction, a steady stream of products is created, one being a chain of hydroxyl radicals that can now exert a more long-lasting effect. This sustained, looped chain reaction also allows for a “shelf life” where the reaction can be maintained and stored for future use even after the radiation exposure has been terminated. An effect that can now be measured in minutes, hours or days due to the continued effect of the reaction products created.

In reference to the discussed reactions, the embodiments explain new discoveries whereby the radiation directed at the oxidizing agent alters the typical reaction. This can be accomplished by excluding wavelengths of radiation that inhibit the formation of trioxygen or wavelengths that destroy trioxygen. This creates and allows trioxygen to function as a photocatalyst.

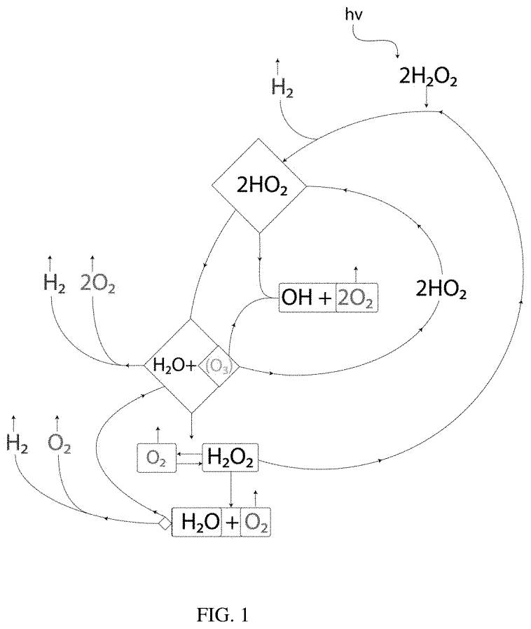

The following embodiment relates to the working model of the equation for the looped chain reaction. In chemical kinetics, an equation dictates that a chemical reaction utilizing oxidizing agents proceeds via a decomposition reaction where an electron induced decomposition by radiation (excluding wavelengths inhibiting trioxygen formation or destroying trioxygen) of the oxidizing agent proceeds. X defines potential decomposition by-products such as reactive nitrogen species, hydroxyls, hydroperoxyls, electronically modified oxygen species, hydrogen and oxygen and others. A reaction can occur from a reactant molecule via an intermediate such as hydroperoxyl to form an trioxygen molecule, as shown in FIG. 1.

OXIDIZING AGENT+radiation dose (excluding wavelengths that dissociate trioxygen (O3))→O3+X.

In reference to the above reactions, this embodiment explains discoveries whereby the radiation directed at the oxidizing agent alters the typical reaction. This can be accomplished by excluding wavelengths of radiation that inhibit the formation of trioxygen or wavelengths that destroy trioxygen. Photochemical reactions are a chemical reaction initiated by the absorption of energy in the form of light. The consequence of molecules' absorbing light is the creation of transient excited states whose chemical and physical properties differ greatly from the original molecules. Photochemicallytic trioxygen generation (PTG) splits water molecules into H2, O2, and O3. PTG can achieve high dissolution in water without other competing gases found in the corona discharge method of trioxygen production, such as nitrogen gases present in ambient air. This method of generation can achieve consistent trioxygen concentration and is independent of air quality because water is used as the source material. Production of trioxygen photochemically was previously not utilized because of the required wavelength exclusion required to produce trioxygen as compared to producing oxygen as the typical reaction product. However, as described herein, it is possible to change the production of oxygen by careful selection of radiation wavelengths such that trioxygen is preferentially produced. Previous research involving UV radiation utilized bulbs that produced a bell-shaped curve of radiation that produced wavelengths of dissociation of compounds and wavelengths creating the same compounds. While there may have been a greater influence of either the creation or dissociation wavelength, the resulting reaction was at best inefficient.

Thus, in the present embodiments, to generate more trioxygen, wavelengths of radiation that dissociate trioxygen are excluded and the dose of radiation can be increased by increasing the intensity, the time the radiation is applied and other variables to the dose where some or all variables may be changed. This data helps to demonstrate the nature of the initial complex which decomposes an oxidizing agent upon radiation exposure. Further, multiple reaction sequences are possible. First, comparing the electronic structure of the water and the oxidizing agent molecules, the trioxygen should cleave at least one oxygen-hydrogen bond of the water molecule in the looped chain reaction, which, in turn, forms the hydroxyl radical plus atomic hydrogen. This process is endoergic. Two of the hydroxyl radicals can recombine in an exoergic reaction to form an oxidizing agent molecule. The reaction reversibility dictates that upon application of trioxygen to the water molecule, the latter can decompose in one step to form oxygen atoms plus molecular hydrogen. The oxygen atom in the presence of trioxygen can react now with a water molecule by an insertion into an oxygen-hydrogen bond to form hydrogen peroxide but with the continued application of trioxygen, the generation of H2O2 may be delayed or excluded. As the reaction is delayed, oxygen and hydrogen may be liberated in sufficient quantities to alter the quantity of available components thus preventing or minimizing the production of H2O2. Alternatively, the oxygen atom can add itself to the oxygen atom of the water molecule forming a short-lived intermediate which rearranges then via hydrogen migration to the hydrogen peroxide molecule. These equations display an electron induced decomposition of two water molecules in close proximity. [(H₂O(X¹A₁))₂] to form a hydrogen peroxide molecule while liberating hydrogen and oxygen.

H₂O(X¹A₁)+TRIOXYGEN→H(²S_{1/2})+OH(X²Π_{Ω}) Equation 13

2OH(X²Π_{Ω})+TRIOXYGEN→H₂O₂(X¹A) Equation 14

H₂O₂(X¹A₁)+TRIOXYGEN→O(¹D)+H₂(X¹Σ_{g}⁻) Equation 15

O(¹D)+H₂O(X¹A₁)+TRIOXYGEN→H₂O₂(X¹A) Equation 16

O(¹D)+H₂O(X¹A₁)+TRIOXYGEN→[OOH₂(X¹A)]+TRIOXYGEN→H₂O₂(X¹A) Equation 17

(A)[(H₂O(X¹A₁))₂]+TRIOXYGEN→[H(²S_{1/2}) . . . HO(X²Π_{Ω}) . . . OH(X²Π_{Ω}) . . . H(²S_{1/2})]+TRIOXYGEN→H₂O₂(X¹A)+2H(²S_{1/2}) Equation 18

(B)[(H₂O(X¹A₁))₂]+TRIOXYGEN→[H₂(X¹Σ_{g}⁺) . . . H₂O(X¹A₁) . . . O(¹D)]+TRIOXYGEN→H₂(X¹Σ_{g}⁺)+H₂O₂(X¹A) Equation 19

(C)[(H₂O(X¹A₁))₂]+TRIOXYGEN→[H₂(X¹Σ_{g}⁺) . . . H₂O(X¹A₁) . . . O(¹D)]+TRIOXYGEN→[H₂(X¹Σ_{g}⁺) . . . H₂OO(X¹A)]+TRIOXYGEN . . . HO3 . . . HO4→H₂(X¹Σ_{g}⁺)+H₂O₂(X¹A) Equation 20

As can be seen above from the equations, the water solution still stores highly reactive radicals such as RNS, EMODs, hydroxyl radicals, hydroperoxyls, and the like. Hydroxyl radicals can diffuse and once they encounter a second hydroxyl radical, they can recombine to form hydrogen peroxide. As described herein, it may be understood that upon a decomposition of the water molecules, the oxygen atoms are formed in the first excited state. When the radiation exposure stops and the trioxygen is depleted, the production of excited atoms ceases, too. This reinforces the fact that, without removing the wavelengths that dissociate trioxygen, this reaction cannot proceed as described. The reactivity of ground state atoms with water is different compared to the dynamics of the trioxygen excited counterparts generated during exposure to trioxygen described in this patent via stated equations. The data and related discussion on the formation of the hydrogen peroxide molecule also help to explain the synthesis of atomic and molecular hydrogen during the trioxygen exposure of the oxidizing agent and/or water or solution or combination of solution composition. Here, the equations indicate that molecular hydrogen can be formed in a one-step mechanism via trioxygen decomposition of the water molecule driven by the trioxygen dose applied to the solution. Alternatively, the hydrogen atoms formed can recombine to form molecular hydrogen. The detection of hydrogen atoms during the trioxygen exposure of the oxidizing agent or water or solution or combination of solution composition phase is a direct proof that the reactions take place. Likewise, the observation of oxygen atoms during the trioxygen exposure suggests that the reactions are also an important pathway of oxygen production. The matrix may store hydrogen as hydronium or other isotopes of hydrogen and as suspended “bubbles” of hydrogen even when the radiation exposure is terminated and trioxygen has ceased to be produced. By placing the matrix in a sealed container so that the suspended gases are not allowed to escape, pressure that builds up maintains the reactivity and this potential can be stored for future use.

Hydroxyl radicals (OH) are formed via a decomposition of a water molecule upon exposure to trioxygen. This trioxygen driven, looped chain reaction, generates hydrogen, oxygen, free radicals as well as oxidizing molecules including however, but not limited to, electronically modified oxygen derivatives from water or solutions containing oxidizing agents that are exposed to radiation which when introduced to an effective amount of a composition comprising water and/or an oxidizing agent compound or other compounds or solutions then exposing the composition to trioxygen, where the composition including the water and/or oxidizing agent compound, solution or both, functions together with trioxygen to lead to a reaction producing hydrogen and/or its isotopes, and/or oxygen and/or its isotopes and/or electronically modifies oxygen derivatives and or solutions derived or indirectly derived resulting from the exposure of the wavelength(s) in the looped chain reactions and the resultant trioxygen used in the looped chain reactions or the synergy therein. Also, it can be shown that there is a decomposition of the HO₂ radical to molecular oxygen plus atomic hydrogen. Finally, to generate the HO₂ radical, another reaction is hydrogen atoms reacting with molecular oxygen but with the application of the correct wavelengths of radiation to the oxidizing agent undergoing this reaction in the looped chain reactions, the excited state of produced hydrogen atoms and the produced molecular oxygen and the generation of trioxygen can be retarded or stopped by the discontinuance of the radiation used. The excited state can be preserved by sealing the reactants so that produced gases are maintained, and this allows for the reactive potential to be stored.

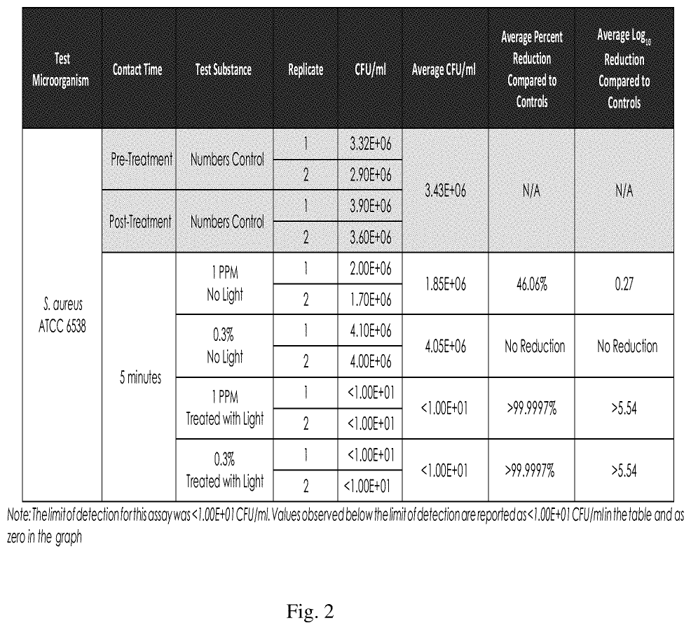

Thus, these embodiments uncover a significant reaction sequence that has not been previously known or understood. By exposing an oxidizing agent to certain doses of radiation, hydrogen is liberated from the reaction. Hydroperoxyls are produced and trioxygen is produced when wavelengths that dissociate trioxygen are eliminated or reduced in intensity. This reaction generates hydrogen, oxygen, trioxygen and other free radicals as well as oxidizing molecules including however not limited to electronically modified oxygen derivatives from oxidizing agents or solutions containing oxidizing agents that are exposed to certain wavelengths of radiation which when introducing an effective amount of a composition comprising an oxidizing agent compound or other compounds or solutions then exposing the composition to radiation of certain wavelengths while excluding the wavelengths that would disallow the formation of trioxygen, wherein the composition comprising the oxidizing agent compound, solution or both, functions together with the radiation of certain wavelength or wavelengths to lead to a reaction producing trioxygen, hydrogen and/or its isotopes, and/or oxygen and/or its isotopes and/or electronically modifies oxygen derivatives and or solutions derived or indirectly derived resulting from the exposure of said wavelength(s) or the synergy therein. The oxidizing potential of trioxygen is slightly less than the oxidizing potential of hydroxyl radicals, but it is greater than the oxidizing potential of hydrogen peroxide. While the commonly accepted lifetime of hydroxyl radicals is a few nanoseconds, trioxygen has been shown to maintain its reactivity for hours. The ability of trioxygen to linger for an extended period allows for a “stored” oxidizing effect. The “stored” oxidizing effect can be tapped to provide reactive oxygen species as needed and the “stored” oxidizing effect feeds the looped chain reaction so that reactive oxygen species are generated until one of the reactants is depleted. FIG. 2 reflects testing that displays this “stored” effect. When comparing the control versus the enhanced solution, there is over a 5-log increase in efficacy with the enhanced solution. By employing the looped chain reactions, we have increased the efficacy and reserved the use of the electronically modified oxygen derivatives that are being continuously generated so that they are available for use over an extended period of time.

The equations are exemplary and are non-limiting with respect to wavelengths, time of irradiation, intensity of radiation or total dose of radiation. By exposing the oxidizing agent or agents to radiation of between 100 nm-1200 nm, a synergistic reaction occurs creating trioxygen and other electronically modified oxygen derivatives and disrupting the typical disassociation reaction of the oxidizing agent or agents. Chemicals such as oxidizing agents exist in a state of flux whereby, they disassociate and reassociate as self-ionization reactions occur. When alterations of the expected disassociation reactions occur, new compounds or variations in compound concentrations occur. These new compounds or variations in compounds created in the synergistic reaction enable a known oxidizing agent to create reactions that have not been observed or reported previously. By restricting the radiation applied to the oxidizing agent so that dissociation of trioxygen is reduced or eliminated, a reaction is produced that has previously not been observed. This is shown by the radiation typically produced as having wavelengths that dissociate trioxygen when said radiation is applied to oxidizing agents. Restricting the dissociation of trioxygen has produced reaction products that have not been described for this reaction previously or that have not been produced in quantities that are shown in this patent application.

The reactants may contain enzymes, stabilizers or other substances that affect the overall reaction rate. Enzymes, stabilizers and/or other substances can be destroyed or inactivated by temperature variations, pH shifts and other means. These may be employed to arrive at the most favorable reaction outcomes. It is understood that phosphoric acid (H₃PO₄) is generally added to commercially available oxidizing agent solutions such as hydrogen peroxide as a stabilizer to inhibit the decomposition of the oxidizing agent. Several types of reagents, such as H₃PO₃, uric acid, Na₂CO₃, KHCO₃, barbituric acid, hippuric acid, urea, and acetanilide, have also been reported to serve as stabilizers for oxidizing agents such as hydrogen peroxide. These stabilizers have been shown to have a catalyst effect on the reaction, but the reaction may proceed with or without stabilizers present in oxidizing agents, as desired.

The embodiments describe new methods and techniques that have not been described or understood previously. By altering the typical disassociation wavelength of radiation applied to oxidizing agents, the ensuing reaction generates previously unrecorded reaction byproducts and/or quantities of byproducts.

The embodiments demonstrate the new discovery of altering the expected reactions found by the disassociation of trioxygen while radiation of certain wavelengths is targeted to oxidizing agents and by so altering the expected disassociation compounds are generated that have not been reported from the typical disassociation reactions. This discovery has applications in many industries. By increasing the efficacy of oxidizing agents, common chemical reactions involving oxidizing agents may be accomplished using less volume and/or a lower concentration of oxidizing agents. Oxidizing agents can be used to precipitate material out of solution. Increasing the efficacy of the oxidizing agent allows for this precipitation with less oxidizing agent. Oxidizing agents have antimicrobial properties. By increasing the antimicrobial efficacy with the methods described herein, concentrations of oxidizing agents utilized can be reduced while efficacy can be maintained or increased. By increasing the availability of ROS in the irradiated oxidizing agent solution, current applications of oxidizing agents in the semiconductor industry, paper industry, petrochemical industry and other commercial applications can be accomplished faster and/or more economically and/or more environmentally responsibly. The uses of the methods described herein are too numerous to list but are widespread in diverse industries from oil and gas to health care and beyond.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

1. A method for generating one or more of hydrogen, isotopes of hydrogen, oxygen, and isotopes of oxygen and for electronically modifying oxygen derivatives, reactive oxygen species (ROS), trioxygen, and free radicals, comprising:
applying to a target liquid having a volume and a pH, at least one oxidizing agent in an amount from less than 1 part per million to 50 percent or more of the volume of the target liquid to produce in the target liquid one or more of hydrogen and its isotopes, and oxygen and its isotopes when exposed to radiation having a wavelength in a range of 300 nanometers through 600 nanometers;
applying radiation having a wavelength in a range of 300 nanometers through 600 nanometers to the at least one oxidizing agent at least before, during, and/or after the at least one oxidizing agent is applied to the target liquid to form a reaction and produce in the target liquid the one or more of hydrogen and its isotopes, and oxygen and its isotopes, which comprise at least trioxygen and hydroxyl radical; and
adjusting the pH of the target liquid to stabilize the trioxygen or react the trioxygen with hydroxyl radical, and
optionally applying enzymes or applying stabilizers to affect the overall reaction rate, the stabilizers being selected from the group consisting of H₃PO₃, uric acid, Na₂CO₃, KHCO₃, barbituric acid, hippuric acid, urea, acetic acid and acetanilide,
wherein wavelengths of 307 nm, 402 nm, 452 nm, and 599 nm, which photodissociate, eliminate, or reduce trioxygen, are excluded from the radiation,
wherein substances that aid in affecting the overall reaction rate are the trioxygen produced by the reaction and the optionally applied enzymes or stabilizers, and
wherein the reaction is able to proceed with or without said enzymes, said stabilizers or other substances that affect the overall reaction rate.

2.-3. (canceled)

4. The method of claim 1, wherein the radiation source is a bulb or LED, and the radiation is applied directly or indirectly to at least one of the oxidizing agent, the trioxygen or the target liquid.

5. The method of claim 1, wherein an oxidizing agent dispenser is used to apply the at least one oxidizing agent to the target liquid, and the oxidizing agent dispenser is a pump, a mister, a diffuser, or an electrostatic sprayer.

6.-7. (canceled)

8. The method of claim 1, wherein the irradiated at least one oxidizing agent is used to precipitate material out of solution.

9. The method of claim 1, wherein the irradiated at least one oxidizing agent is used as an antimicrobial agent.

10. The method of claim 1, wherein the irradiated at least one oxidizing agent is used as a bleaching agent.

11. (canceled)

12. The method of claim 1, wherein the radiation is applied to the at least one oxidizing agent before the at least one oxidizing agent is applied to the target liquid, the target liquid furthers a photo oxidization reaction that generates one or more of hydrogen and its isotopes, and oxygen and its isotopes and electronically modifies oxygen derivatives, ROS, trioxygen, and free radicals or produces additional reactions, and the further or additional reactions are not dependent on continued or additional exposure of radiation.

13. The method of claim 1, wherein the radiation is applied to the at least one oxidizing agent after the at least one oxidizing agent is applied to the target liquid so that the trioxygen and the one or more of hydrogen and its isotopes, and oxygen and its isotopes are generated after the at least one oxidizing agent is applied to the target liquid, and a photo oxidation reaction is not initiated.

14. The method of claim 13, wherein the generating of the one or more of hydrogen and its isotopes, and oxygen and its isotopes and electronically modifying oxygen derivatives, ROS, trioxygen, and free radicals produces a desired effect at a predetermined time after application of the at least one oxidizing agent to the target liquid, and the predetermined time is variable.

15. The method of claim 1, wherein the generating of one or more of hydrogen and its isotopes, and oxygen and its isotopes and electronically modifying oxygen derivatives, ROS, trioxygen, and free radicals occurs in a sealed container whereby gases created by the generating of the one or more of RNS, hydrogen and its isotopes, and oxygen and its isotopes and electronically modifying oxygen derivatives, ROS, trioxygen, and free radicals are not allowed to escape.

16. The method of claim 1, wherein the at least one oxidizing agent is selected from Oxygen (O₂), trioxygen (O₃), Hydrogen (H), Hydrogen peroxide (H₂O₂) or other inorganic peroxides, Fenton's reagent, Fluorine (F₂), chlorine (Cl₂), or other halogens, Nitric acid (HNO₃) or nitrate compounds, Sulfuric acid (H₂SO₄), Peroxydisulfuric acid (H₂S₂O₈), Peroxymonosulfuric acid (H₂SO₅), or other Sulfur compounds, Hypochlorite, Chlorite, chlorate, perchlorate, or other analogous halogen compounds, chromic or dichromic acids, chromium trioxide, pyridinium chlorochromate (PCC), chromate, or dichromate compounds, or other hexavalent chromium compounds, potassium permanganate (KMnO₄), Sodium perborate, or other Permanganate compounds, Nitrous oxide (N₂O), Nitrogen dioxide/Dinitrogen tetroxide (NO₂/N₂O₄), urea, Potassium nitrate (KNO₃), Sodium bismuthate (NaBiO₃), ceric ammonium nitrate, ceric sulfate, or other Cerium (IV) compounds, peracetic acid, and Lead dioxide (PbO₂).

17. The method of claim 1, further comprising selecting the at least one oxidizing agent dependent on properties of whether the target liquid to be treated is under aerobic or anaerobic conditions, the pH of the target liquid, temperature of the target liquid, salinity of the target liquid, consortium or population characteristics of organisms or micro-organism present, content of the target liquid, content of any biofilms associated with the target liquid or otherwise a composition on the target liquid.

18. The method of claim 1, wherein the at least one oxidizing agent further comprises at least one other substance for a desired process includes antimicrobial properties, anticorrosion properties, anti-neoplastic properties, thermal properties, explosive properties, precipitation properties, electrochemical properties, power generation properties or other desired effects obtained in combination with the desired process.

19. (canceled)

20. A system configured to perform the steps of the method of claim 1, comprising:
a reaction area, in which the at least one oxidizing agent functions together with the radiation of certain wavelengths to lead to a synergistic reaction, so that the products of the reaction can be collected and separated any time during the reaction if desired,
at least one oxidizing agent introducing component for applying the at least one oxidizing agent to the target, and
at least one radiation emitting component for creating the radiation wherein wavelengths that can photodissociate, eliminate, or reduce trioxygen are excluded from the radiation.

21. The system of claim 20, further comprising:
at least one or more sensors or other devices to indicate, detect, or inform of one or more of the following properties of the target or storage or environment:
pH, temperature, salinity, density, trioxygen concentration, oxygen concentration, hydrogen concentration, oxidizing agent concentration, flow rate, microbial content, presence or absent of bacterial species, presence or absent of corrosive metabolites or otherwise corrosive substance, identification of a gas, presence or absent of an aqueous environment, presence or absent of high, low, or otherwise concentration of bacterial or non-bacterial, biomass or non-biomass, microbial content, or location of biofilms.

22. The system of claim 20, wherein the at least one radiation emitting component emits, delivers, produces, or otherwise facilitates the radiation between 100 nanometers and 1200 nanometers, independently, simultaneous, continuously, or intermittently.

23. The system of claim 20, wherein the at least one radiation emitting component is suspended, adjacent to, inside of, surrounding or associated with a container, structure or area of the at least one oxidizing agent, or the target or supported in a target container, so that the at least one radiation emitting component is physically close to the at least one oxidizing agent and/or the target.

24. The system of claim 20, wherein the at least one radiation emitting component adjusts the radiation wavelengths, intensity, duration or location relative to the target on the basis of any one or more of the density and light absorbing or reflection quality of the target to be treated, the size, shape, or composition of the reaction area, conditions or properties of the environment, whether the target is under aerobic or anaerobic conditions, pH, temperature, salinity of the target, consortium or population characteristics of any organisms or micro-organisms present in the target, the microbial content of the target, and the microbial content of any biofilm present in the target, the reaction area, or the environment.

25. The system of claim 20, wherein concentration, temperature, viscosity, PH and/or other variables of the at least one oxidizing agent are adjusted to produce the desired reaction or results.

26. The system of claim 20, wherein the at least one oxidizing agent and the target is a liquid, solid, gas, plasma or combination thereof, either independently or simultaneously.

27. The system of claim 20, wherein the reaction is affected or initiated by the addition of other catalysts.

28. The system of claim 20, wherein the radiation is directly or indirectly applied to the at least one oxidizing agent, the target, or the combination thereof, and the indirect application is application by fiber optics cable, reflection, or other means of transmission.

29. The system of claim 20, wherein the duration of the radiation includes less than 1 second, greater than 1 second, continuous, pulsed, or intermittent.

30. The system of claim 1, wherein the at least one oxidizing agent is heated or cooled to activate and/or inactivate enzymes present in the target.

31. The method according to claim 1, wherein the method is performed without applying enzymes, stabilizers or other substance that affect the overall reaction rate.

32. The method according to claim 1, wherein the method is performed by applying radiation having a wavelength in a range of greater than 307 nm to less than 599 nm with wavelengths of 402 nm and 452 nm being excluded.

33. The method according to claim 16, wherein the at least one oxidizing agent is hydrogen peroxide.
